(19) United States
(12) Patent Application Publication
(10) Pub. No.: US 2025/0160771 A1
(43) Pub. Date: May 22, 2025

(54) X-RAY IMAGING APPARATUS

(71) Applicants: RAYENCE Co., Ltd., Gyeonggi-do (KR); VATECH EWOO Holdings Co., Ltd., Gyeonggi-do (KR); Qpix solutions Inc., Morrisville, NC (US)

(72) Inventors: Seungman YUN, Morrisville, NC (US); Changhyeuk KIM, Morrisville, NC (US); Byeongnyeon KIM, Gyeonggi-do (KR); Jongbo YOON, Gyeonggi-do (KR)

(73) Assignees: RAYENCE Co., Ltd., Gyeonggi-do (KR); VATECH EWOO Holdings Co., Ltd., Gyeonggi-do (KR); Qpix solutions Inc., Morrisville, NC (US)

(21) Appl. No.: 18/951,532

(22) Filed: Nov. 18, 2024

Related U.S. Application Data
(60) Provisional application No. 63/599,796, filed on Nov. 16, 2023.

(51) Int. Cl. A61B 6/00; A61B 6/02

(52) U.S. Cl. CPC A61B 6/4405; A61B 6/4441; A61B 6/4476; A61B 6/025

(57) ABSTRACT

Proposed is a C-arm X-ray imaging apparatus that has a limited footprint to be able to operate and be moved in a narrow space such as an operating room and a treatment/procedure room. The X-ray imaging apparatus includes a C-arm part including a C-shaped arm, and an X-ray generator and an X-ray detector disposed oppositely at both ends of the C-shaped arm, a main body part including a movable mobile base, and an arm part configured to connect the main body part and the C-arm part, wherein the X-ray generator is separately movable with respect to an end of the C-shaped arm, and a footprint of the mobile base is less than 1 m².

### CROSS REFERENCE TO RELATED APPLICATION

This application claims the benefit of the filing date of U.S. Patent Provisional Application No. 63/599,796, filed Nov. 16, 2023, as Attorney Docket No.: 810.0156prov, the teaches of which are incorporated herein their entirety by reference.

### BACKGROUND

### Technical Field

The present disclosure relates to an X-ray imaging apparatus in which an X-ray generator and an X-ray detector are respectively disposed at opposite ends of a C-shaped arm.

### Description of the Related Art

An X-ray machine is a device that emits X-rays to a subject, obtains an X-ray image from the X-rays that travel through the subject to display the internal structure of the subject.

An example of the X-ray machine is an X-ray fluoroscopy apparatus, commonly called a C-arm, which is used in operating rooms, emergency rooms, or during other clinical procedures. As an example, the C-arm X-ray machine consists of a mobile base, a multi-joint arm connected to the mobile base, and a C-shaped arm connected to the multi-joint arm. Additionally, an X-ray generator and an X-ray detector are respectively installed at opposite ends of the C-shaped arm to capture an X-ray fluoroscopy image of an object placed therebetween.

The C-arm X-ray machine is mainly used to acquire two-dimensional X-ray fluoroscopy images of a subject. Meanwhile, in order to check the exact location of a lesion, surgical progress, location of a surgical instrument, and degree of fastening during surgery, tomographic images may be required. However, it is difficult to move a patient to an imaging room equipped with a CT imaging system during surgery to obtain tomography images. A C-arm X-ray machine with a tomosynthesis function, which involves acquiring multiple tomographic images of an object by taking X-rays from different angles within a limited angular range can be an alternative.

The problem is that, to obtain a tomographic image of tomosynthesis, the X-ray generator must rotate and move within a certain angle range to obtain multiple projection images of a subject. In a typical C-arm X-ray machine, because an X-ray generator is fixed to one end of a C-shaped arm, the entire C-shaped arm needs to be rotated to obtain multiple projection images in a certain angle range. In the case of a conventional C-arm X-ray machine with a large and complex driving mechanism, the imaging time is long, precise control is difficult, and due to increased load and volume, it is often difficult to use the machine in an operating room or even get the machine into the operating room.

### DOCUMENT OF RELATED ART

(Patent Document 1) Korean Patent Application Publication No. 10-2016-0071938
(Patent Document 2) Korean Patent Application Publication No. 10-2013-0058633
(Patent Document 3) U.S. Patent Application Publication No. 2020-0085390

### SUMMARY

Accordingly, the present disclosure has been made keeping in mind the above problems occurring in the related art, and the present disclosure is intended to provide a C-arm X-ray imaging apparatus that does not expose the movement of a drive system including an X-ray generator during tomosynthesis, and that has a limited footprint so as to operate and be moved in a narrow space such as an operating room and a treatment/procedure room.

In order to achieve the above objective, according to an aspect of the present disclosure, there is provided an X-ray imaging apparatus including: a C-arm part including a C-shaped arm, and an X-ray generator and an X-ray detector disposed oppositely at both ends of the C-shaped arm; a main body part including a movable mobile base; and an arm part configured to connect the main body part and the C-arm part, wherein the X-ray generator may be separately movable with respect to an end of the C-shaped arm, and a footprint of the mobile base may be less than 1 m².

The footprint may be within 0.72 m².

The apparatus may further include a C-arm supporter configured to connect the arm part and the C-shaped arm, wherein the arm part may be connected to the main body part and the C-arm supporter through a first axis and a second axis, respectively, and the first and second axes may remain parallel with respect to movement of the arm part relative to the first axis.

The C-shaped arm may be capable of sliding relative to the C-arm supporter.

The C-arm part may include: a guide rail connected to the end of the C-shaped arm; a driving part configured to include a motor and to move the X-ray generator back and forth along a length direction of the guide rail by rotating the motor; and a casing installed at the end of the C-shaped arm, and configured to cover the X-ray generator, the guide rail, and the driving part.

The guide rail may be arch-shaped, and the X-ray generator may move back and forth along the guide rail while maintaining an equal distance from the X-ray detector.

The apparatus may further include a controller provided in the main body part and configured to reconstruct an X-ray image based on an X-ray detection result of the X-ray detector, wherein the X-ray generator may move separately with respect to the end of the C-shaped arm and capture tomosynthesis images from different angles for a subject placed between the X-ray generator and the X-ray detector, and the controller may reconstruct a plurality of tomography images of the subject using the tomosynthesis imaging results.

A front end of the C-arm part may extend forward from the mobile base to a length of 1150 mm or more.

According to the present disclosure, provided is a C-arm X-ray imaging apparatus that does not expose the movement of a drive system including an X-ray generator during tomosynthesis, and that has a limited footprint to be able to operate and be moved in a narrow space such as an operating room and a treatment/procedure room.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a side view of an X-ray imaging apparatus according to an embodiment of the present disclosure with a C arm extended forward;

FIG. 2 is a top view of the X-ray imaging apparatus shown in FIG. 1;

FIG. 3 is a side view of the folded state of the X-ray imaging apparatus according to an embodiment of the present disclosure;

FIG. 4 is a top view of the X-ray imaging apparatus shown in FIG. 3;

FIG. 5 is a side view showing a generator part casing removed from the X-ray imaging apparatus according to an embodiment of the present disclosure; and

FIG. 6 is a perspective view of the X-ray imaging apparatus shown in FIG. 5.

### DETAILED DESCRIPTION

Hereinafter, embodiments of the present disclosure will be described in detail with reference to the drawings. The technical idea of the present disclosure may be more clearly understood through the embodiments. The present disclosure is not limited to the embodiments described below.

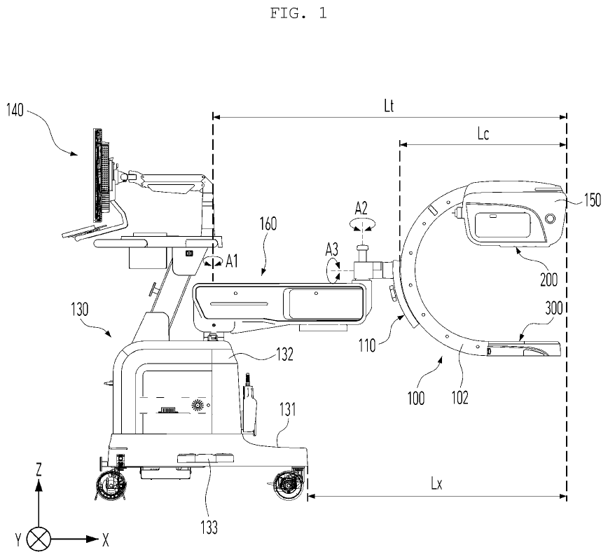

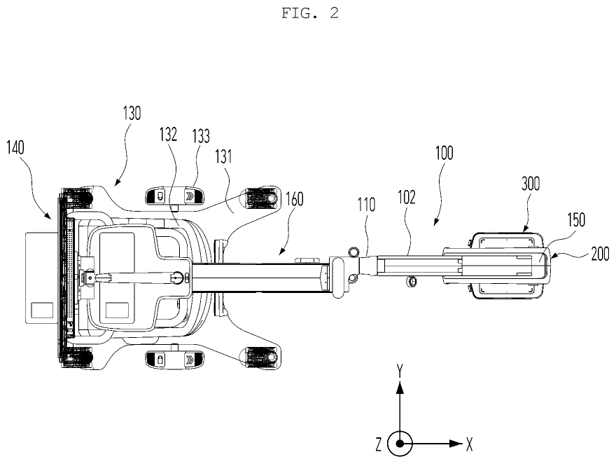

FIG. 1 is a side view of an X-ray imaging apparatus according to an embodiment of the present disclosure with a C arm extended forward. FIG. 2 is a top view of the X-ray imaging apparatus shown in FIG. 1.

In FIGS. 1 and 2 and the following drawings, the x-axis direction represents the front of the X-ray imaging apparatus, the y-axis direction represents a direction perpendicular to the x-axis on the plane where the X-ray imaging apparatus is placed, and the z-axis direction represents a direction perpendicular to the plane.

Referring to FIGS. 1 and 2, the X-ray imaging apparatus according to an embodiment of the present disclosure largely include a main body part 130, a C-arm part 100, and an arm part 160 connecting the main body part 130 and the C-arm part 100. A mobile base 131 is provided at the lower part of the main body part 130 to support the entire load of the apparatus and to be movable. Additionally, the main body part 130 may have a built-in controller such as a console PC responsible for apparatus control and image processing. The controller may be disposed on the upper part of the main body part 130 and connected to a display 140 that outputs an X-ray image, etc., and an input part through which user manipulation is input.

The mobile base 131 may include a plurality of wheels and a frame supporting the wheels for stable movement. In addition, when viewed from above as shown in FIG. 2, in the mobile base 131, the area between the parts supporting the two front wheels of the frame is concavely recessed toward a body 132 of the main body part 130 to reduce interference with the feet of a user moving or operating the apparatus. A reference numeral 133 denotes a locking pedal for fixing the wheels.

The rear end of the arm part 160 is connected to the main body part 130 and the front end of the arm part 160 is connected to the C-arm part 100. To be specific, the arm part 160 is connected to the main body part 130 so as to rotate up, down, left, and right within at least a predetermined angle range about a first axis A1 parallel to the z-axis. The front end of the arm part 160 and the C-arm part 100 are connected through a C-arm supporter 110.

The C-arm part 100 includes an X-ray generator 200 (hereinafter referred to as generator), an X-ray detector 300 (hereinafter referred to as detector), and a C-shaped arm 102 with the X-ray generator 200 and the X-ray detector 300 respectively disposed at opposite ends.

The C-arm supporter 110 has one side thereof supporting the C-arm part 100 so as to slide along the C-shaped curved surface of the C-shaped arm 102, and has the other side thereof rotatably connected to the front end of the arm part 160 about a second axis A2. One side of the C-arm supporter 110 supporting the C-arm part 100 may be rotatably connected to the other side connected to the arm part 160 about a third axis A3 perpendicular to the second axis A2.

The first axis A1 may be connected to the upper part of the body 132 of the main body part 130 so that the arm part 160 may rotate left and right with respect to the main body part 130. In addition, the rear end of the arm part 160 may be configured to be rotatable up and down about an axis perpendicular to the first axis A1 so that the height of the C-arm part 100 connected to the opposite side may be adjusted. The body 132 is disposed biased toward the rear from the center of the mobile base 131, and the first axis A1 may be disposed on the upper surface of the body 132 when viewed from above. This arrangement is advantageous for maintaining the center of gravity within the footprint of the mobile base 131 and allowing the C-arm part 100 to fully extend forward. Such arrangement is also advantageous in reducing the space occupied when the C-arm part 100 and the arm part 160 are folded for storage or movement.

Due to the above configuration, in the X-ray imaging apparatus according to the present disclosure, when an anteroposterior length Lc of the C-arm part 100 is 500 to 900 mm, for example, 698 mm, and a straight line distance Lt from the front end of the C-arm part 100 to the first axis A1 on the rear end side of the arm part 160 is 1200 to 1700 mm, for example, 1481 mm, a maximum distance Lx that the C-arm part 100 reaches forward from the front end of the mobile base 131 is 1000 to 1300 mm, for example, 1150 mm.

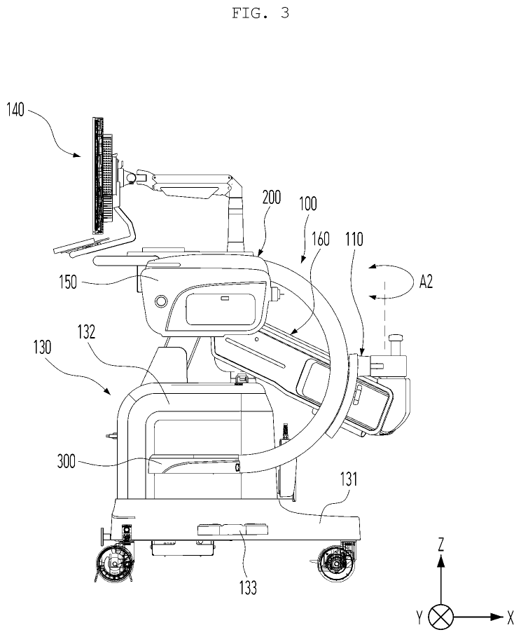

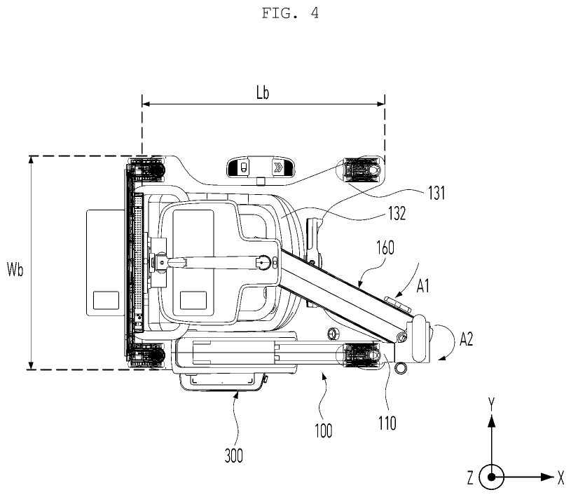

FIG. 3 is a side view of the folded state of the X-ray imaging apparatus according to an embodiment of the present disclosure. FIG. 4 is a top view of the X-ray imaging apparatus shown in FIG. 3.

When moving or storing the X-ray imaging apparatus according to the present disclosure, as shown, the arm part 160 and the C-arm part 100 may be transformed into a folded state in which the arm part 160 and the C-arm part 100 are folded to the side of the main body part 130 and are in close contact. At this time, the arm part 160 and the C-arm part 100 rotate about the first axis A1 and the second axis A2, respectively, so that the C-arm part 100 is located on the side of the body 132 of the main body part 130. The C-arm part 100 may be configured to be temporarily fixed to the side of the body 132 by using a magnet or other temporary fixing means provided on the body 132. In this case, the direction in which the arm part 160 and the C-arm part 100 are folded is not limited, and may be configured to be folded on both left and right sides.

This folded posture lowers the center of gravity of the entire apparatus, adding stability during movement and storage, and reduces the extent to which the arm part 160 and the C-arm part 100 protrude out of the mobile base 131, providing the effect of greatly reducing the volume during movement and storage.

The X-ray imaging apparatus according to the present disclosure may be configured so that the footprint does not exceed 1 m², preferably not more than 0.72 m². A front-back length Lb of the mobile base 131 may be 700 to 1,000 mm, for example, 839 mm, and a width Wb may be 500 to 1,000 mm, for example, 740 mm.

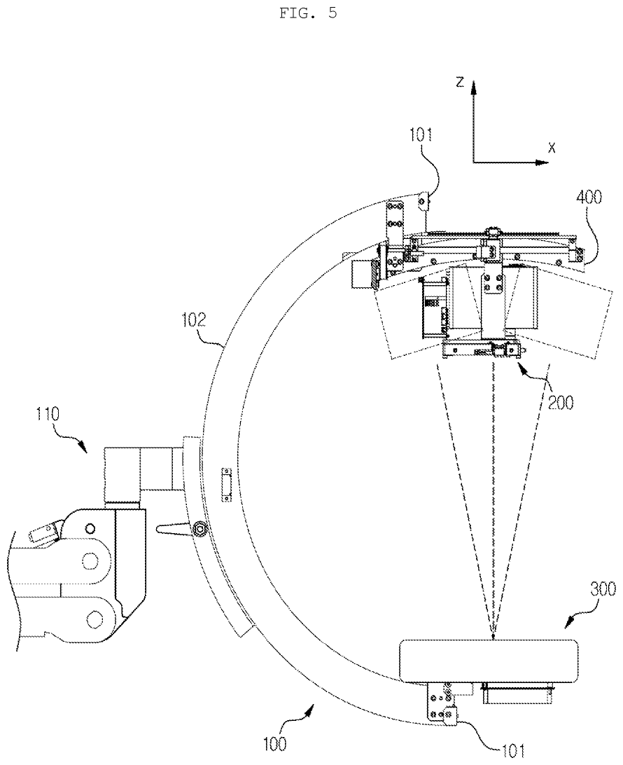

FIG. 5 is a side view showing a generator part casing removed from the X-ray imaging apparatus according to an embodiment of the present disclosure.

FIG. 5 shows a generator part casing 150 removed from the position shown in FIG. 1 so that the principle of tomosynthesis imaging using the X-ray imaging apparatus according to this embodiment can be understood. The C-shaped arm 102, which constitutes the framework of the C-arm part 100, may slide along a C-shaped bend with respect to the C-arm supporter 110, and a stopper 101 is provided at each end of the C-shaped arm 102 to limit the range of sliding.

In the C-arm part 100, an arcuate guide rail 400 is disposed at one end of the C-shaped arm 102 in the front-back direction of the extension line, the generator 200 is arranged to move in the front-back direction along the arcuate guide rail 400. In FIG. 5, the outline of the generator 200 indicated by a thin dotted line represents the maximum range over which the generator 200 may move, and the thick dotted line indicates the change in the irradiation angle of an X-ray beam when the generator 200 is located at the front/rear ends and center of the arcuate guide rail 400. As the detector 300 receives X-rays that pass through a subject (not shown) located between the generator 200 and the detector 300 at various angles, multiple projection images may be acquired within a limited angular range and from these, tomosynthesis images, which are multiple tomographic images of the subject, may be reconstructed.

In addition, as the generator 200 moves along the arcuate guide rail 400 connected to one end of the C-shaped arm 102 for tomosynthesis imaging, the mechanical configuration of the C-arm part 100 may be simplified compared to the case where the entire C-arm part 100 moves. Thus, the total load of the C-arm part 100 may be 30 kg or less, for example, about 23 kg, and the total weight of the X-ray imaging apparatus may be 180 to 220 kg, for example, about 200 kg. As a result, the X-ray imaging apparatus according to the present disclosure has a relatively small footprint area, and the C-arm part 100 may be extended forward from the front end of the mobile base 131 to reach 1000 to 1500 mm, for example, 1150 mm, and stable tomosynthesis imaging is possible in the extended state.

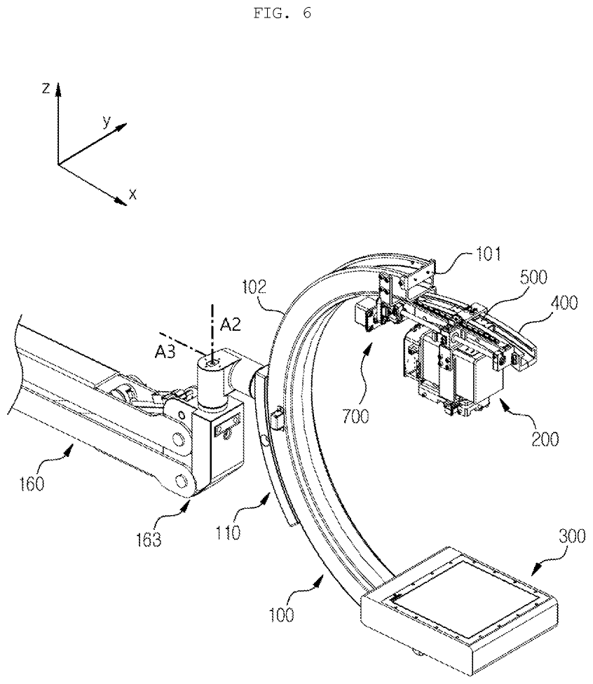

FIG. 6 is a perspective view of the X-ray imaging apparatus shown in FIG. 5.

Through FIG. 6, the driving mechanism of the generator 200 may be examined in more detail. The X-ray imaging apparatus according to the present disclosure includes a driving part 700 that provides power to move the generator 200. The driving part 700 may include a motor. In addition, the driving part 700 converts the rotational motion of the motor into linear motion and transmits the linear motion back to a moving block 500 disposed within the arcuate guide rail 400, so that the moving block 500 may move in an arc-shaped trajectory along the arcuate guide rail 400. The generator 200 is fixed to the moving block 500 and moves along the arcuate guide rail 400. As a result, the generator maintains an equal distance from the detector and may be moved within a predetermined angle range. However, the above-described power transmission method is not limited and may be implemented in various ways, such as directly transmitting the power of the drive motor to the motion of an arc-shaped trajectory using a flexible power transmission means such as a chain or timing belt.

In addition, in FIG. 6, it is shown that a portion of the front side of the arm part 160 has also had the casing thereof removed. As shown, the arm part 160 may be configured to include a pair of links that are parallel to each other and have the same length, so that the first axis A1 and the second axis A2 at opposite ends of the arm part 160 (see FIG. 1) are always maintained parallel to each other even if the angle of the arm part 160 changes.

What is claimed is:

1. An X-ray imaging apparatus comprising:
a C-arm part comprising a C-shaped arm, and an X-ray generator and an X-ray detector disposed oppositely at both ends of the C-shaped arm;
a main body part comprising a movable mobile base; and
an arm part configured to connect the main body part and the C-arm part,
wherein the X-ray generator is movable in a predetermined range in the forward and backward direction from an end of the C-shaped arm, and
a footprint of the mobile base is less than 1 m².

2. The apparatus of claim 1, wherein the footprint is within 0.72 m².

3. The apparatus of claim 1, further comprising:
a C-arm supporter configured to connect the arm part and the C-shaped arm,
wherein the arm part is connected to the main body part and the C-arm supporter through a first axis and a second axis, respectively,
the C-shaped arm is rotatably connected to the C-arm supporter about a third axis perpendicular to the second axis,
the first and second axes remain parallel with respect to movement of the arm part relative to the first axis, and
the C-shaped arm is capable of sliding relative to the C-arm supporter.

4. The apparatus of claim 1, wherein the C-arm part comprises:
a guide rail connected to the end of the C-shaped arm;
a driving part configured to include a motor and to move the X-ray generator back and forth along a length direction of the guide rail by rotating the motor; and
a casing installed at the end of the C-shaped arm, and configured to cover the X-ray generator, the guide rail, and the driving part.

5. The apparatus of claim 4, wherein the guide rail is arch-shaped, and
the X-ray generator moves back and forth along the guide rail while maintaining an equal distance from the X-ray detector.

6. The apparatus of claim 1, further comprising:
a controller provided in the main body part and configured to reconstruct an X-ray image based on an X-ray detection result of the X-ray detector,
wherein the X-ray generator moves separately with respect to the end of the C-shaped arm and captures tomosynthesis images from different angles for a subject placed between the X-ray generator and the X-ray detector, and
the controller reconstructs a plurality of tomography images of the subject using the tomosynthesis imaging results.

7. The apparatus of claim 1, wherein a front end of the C-arm part extends forward from the mobile base to a length of 1150 mm or more.
